(19) United States
(12) Patent Application Publication
(10) Pub. No.: US 2018/0289924 A1
(43) Pub. Date: Oct. 11, 2018

(54) CATHETERS FOR DEBONDING FOULING AGENTS FROM AN INTERIOR SURFACE THEREOF AND RELATED METHODS

(71) Applicant: Duke University, Durham, NC (US)

(72) Inventors: Vrad W. Levering, Durham, NC (US); Changyong Cao, Durham, NC (US); Gabriel P. Lopez, Durham, NC (US); Xuanhe Zhao, Durham, NC (US); Howard Levinson, Durham, NC (US)

(21) Appl. No.: 15/769,592

(22) PCT Filed: Oct. 28, 2016
(86) PCT No.: PCT/US16/59439

§ 371 Date: Apr. 19, 2018

Related U.S. Application Data
(60) Provisional application No. 62/247,448, filed on Oct. 28, 2015.

(51) Int. Cl. A61M 25/00

(52) U.S. Cl. CPC A61M 25/0017; A61M 25/0032; A61M 2025/004; A61M 2025/0035; A61M 2025/0036; A61M 2025/0019

(57) ABSTRACT

Catheters for debonding fouling agents from an interior surface thereof and related methods are disclosed. According to an aspect, a catheter includes a lumen defining a flexible, interior surface that extends substantially along a length of the lumen for contacting a biological material. The catheter also includes cavities extending along the length and positioned within the lumen adjacent to the surface. The cavities each define a cavity opening. The catheter also includes an inflation hub defining hub openings connected to respective cavity openings. The inflation hub defines a pump port configured to interface with a pump. The inflation hub defines one or more fluid pathways that extend between the hub openings and the pump port for permitting flow of gas between the pump and the cavities.

### CROSS REFERENCE TO RELATED APPLICATION

This application claims the benefit of U.S. Provisional Patent Application No. 62/247,448, filed Oct. 28, 2015, and titled INTERNAL SHAFT FOR CATHETER AND METHODS OF USE, the disclosure of which is incorporated herein by reference in its entirety.

### STATEMENT AS TO FEDERALLY SPONSORED RESEARCH

This invention was made with the support of the United States government under Federal Grant Nos. N0014-13-1-0828 and DMR-1121107 awarded by the U.S. Office of Naval Research (ONR) and the National Science Foundation, respectively. The government has certain rights in the invention.

### TECHNICAL FIELD

The present subject matter relates to catheters. More particularly, the present subject matter relates to catheters for debonding fouling agents from an interior surface thereof and related methods.

### BACKGROUND

Infection associated with the use of urinary catheters is a pervasive and challenging issue in healthcare. There are over 30 million urinary catheters used annually in the United States, and catheter-associated urinary tract infections (CAUTIs) are the most common type of nosocomial infections, which account for 30-40% of all hospital infections and lead to over 50,000 deaths each year. Microbes, such as bacteria colonize the surface of urinary catheters very quickly and often form biofilms in the drainage lumen of catheters. The formation of asymptomatic biofilms in urinary catheters promotes the development of symptomatic CAUTIs, and nearly all patients that undergo catherterization for longer than 28 days will suffer some form of infection. In addition, CAUTIs also contribute to the alrming general increase in antibiotic resistance due to horizontal gene transfer between bacteria within biofilms, and the frequent use of antibiotics in their treatment.

Current commercially marketed strategies, such as killing bactiera or delaying bacterial attachment to reduce infection induced by urinary catheters have been unsuccessful in the long-term prevention of biofilm formation which ultimately leads to CAUTIs. Although recent research on techniques to prevent catheter infection, such as bacterial interference and phage delivery show some promise, the are effective only against specific bacterial strains which prohibitively increases the difficulty of their implementation. Identification of the infecting strains is not a typical clinical approach, and even more challenging is the huge variety of infectious microbes, both bacterial and fungal. Indeed, event the most recently discovered new antibiotic is only effective on Gram positive bacteria. Microtopography, permanently attached silicone oils, hydrogels, polymer brushes, and ultrasound are other promising non-strain-specific strategies, but they only delay biofilm formation for a short period and eventually a biofilm still forms. Moreover, the possible large cost to implement them are a hindrance to their routine implementation in clinical settings.

In view of the foregoing, there is a need for improved techniques for removing biofilms from catheters.

### SUMMARY

Disclosed herein are catheters for debonding fouling agents from an interior surface thereof and related methods. According to an aspect, a catheter includes a lumen defining a flexible, interior surface that extends substantially along a length of the lumen for contacting a biological material. The catheter also includes cavities extending along the length and positioned within the lumen adjacent to the surface. The cavities each define a cavity opening. The catheter also includes an inflation hub defining hub openings connected to respective cavity openings. The inflation hub defines a pump port configured to interface with a pump. The inflation hub defines one or more fluid pathways that extend between the hub openings and the pump port for permitting flow of gas between the pump and the cavities.

According to an aspect, a catheter may include a rigid structure positioned between the lumen and cavities. The rigid structure may be tubular in shape. More particularly for example, the rigid structure is positioned inside the hub portion of the shaft in order to prevent over-inflation in the hub portion of the catheter shaft while still allowing flow through a main lumen.

### BRIEF DESCRIPTION OF THE DRAWINGS

The foregoing aspects and other features of the present subject matter are explained in the following description, taken in connection with the accompanying drawings, wherein:

FIGS. 1A-1C illustrate graphs showing stress-strain curves for prototype materials obtained from uniaxial tensile testing;

FIGS. 2A-2C illustrate views of an inflation hub and its configuration with a catheter in accordance with embodiments of the present disclosure;

FIGS. 3A-3C illustrate diagrams of example setups for biofilm-growth and debonding in urinary catheter prototypes;

FIG. 3D is a cross-sectional end view of a catheter as configured within a hub (not shown for ease of illustration) in accordance with embodiments of the present disclosure;

4A-4D illustrate a flow diagram of example use of a urinary catheter for on-demand removal of infectious biofilms via active deformation in accordance with embodiments of the present disclosure;

FIGS. 5A-5F illustrate finite element models and graphs in accordance with embodiments of the present disclosure;

FIG. 6 illustrates a contour plot of nominal strains, and the resultant deformation profile, of the cross-section of a catheter with two lumens when one lumen is actuated;

FIGS. 7A and 7B show finite element analysis and experimental data of a four-lumen catheter shaft made of 50 durometer silicone elastomer;

FIGS. 8A and 8B show experimental testing of a catheter that agrees well with numerical prediction of strain in a central luminal surface as a function of inflation pressure;

FIGS. 9A-9C show representative optical images of the cross sections of control urinary cathether shaft with mixed community P. mirabilis and E. coli biofilm intact on the main lumen of a control versus an actuated catheter;

FIGS. 10A and 10B are graphs showing a storage modulus and loss modulus of biofilm and the silicone substrate as a function of frequency;

FIGS. 11A-11D show the shear forces measured for a control and an experiment;

FIGS. 12A and 12B show the representative optical images from cross sections that were crystal violet stained to enhance visualizations;

FIG. 12C is a graph showing that inflation removed a significant fraction of re-grown biofilm mass in each run;

FIG. 13A shows a control catheter with no inflation;

FIG. 13B shows a first round of inflation after 30 hours of growth of biofilm;

FIG. 13C shows a second round of debonding after re-growing the biofilm for another 24 hours;

FIG. 13D shows sections taken from the prototypes at the following locations: bottom, middle, top, and distal tip;

FIG. 14A shows the strain predicted by finite element models to have occurred in a catheter inflated to 100 kPa;

FIG. 14B shows an optical image of a sliced-open crystal violet stained section of a catheter shaft that experienced two rounds of biofilm growth and debonding;

FIG. 14C shows an optical image of a luminal surface excised from catheter and flattened;

FIG. 14D shows an optical microscopic image of a luminal surface overlying the boundary between the wall and the inflation lumen;

FIGS. 15A and 15B are graphs showing finite element analysis and experimental data of an extruded four-lumen catheter shaft made of 35 durometer silicone elastomer;

FIGS. 16A-16F show deformation profiles from a finite element model of an extruded four-lumen catheter shaft made of 35 durometer silicone shaft and with a 65 durometer silicone sheath when it is subjected to a range of pressures; and

FIG. 17 illustrates an end view of an example lumen shaft 1700 and a mating manifold 1702 in accordance with embodiments of the present disclosure.

### DETAILED DESCRIPTION

For the purposes of promoting an understanding of the principles of the present disclosure, reference will now be made to various embodiments, and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless otherwise defined, all technical terms used herein have the same meaning as commonly understood by one of ordinary skill in the art to which this disclosure belongs.

In describing various embodiments of the present disclosure illustrated in the drawings, specific terminology is employed for the sake of clarity.

However, the presently disclosed subject matter is not intended to be limited to the specific terminology so selected, and it is to be understood that each specific element includes all technical equivalents that operate in a similar manner to accomplish a similar purpose.

The presently disclosed subject matter provides techniques and devices for actively and effectively detaching micro- and macro-fouling organisms through dynamic change of surface area and topology of elastomers in response to external stimuli. These dynamic surfaces can be fabricated from materials used in medical devices and can be actuated by electrical and pneumatic stimulation. New antifouling management strategies based on active surface deformation can also be used in combination with other existing and emerging management approaches for biofouling.

In accordance with embodiments of the present disclosure, a structure is provided that can prevent the adherence of, or allows for the removal of, a fouling agent when exposed to an aqueous environment. As used herein, the term “fouling agent” refers to the undesirable accumulation of microorganisms, plants, algae, and/or animals on a wetted surface. Also within the scope of the presently disclosed subject matter, the term “fouling agent” may refer to the accumulation of a desired cell type, prokaryotic or eukaryotic, that one would want to recover from a surface after it has been accumulated. Examples of such fouling agents include, but are not limited to, bacterial accumulations or other such films desired for biochemical analysis, fungal or other such accumulations used in biotechnology, or accumulations of mammalian cells used in regenerative medicine or other medical procedures or research. The structure comprises, consists of, or consists essentially of a soft polymer layer and an actuation means, wherein the actuation means is capable of deforming the soft polymer layer beyond the critical strain for debonding (□_{c}) of the fouling agent.

The applications of the presently disclosed subject matter include such applications as, for example, debonding of a number of biological films and adsorbates including those formed by culture of mammalian cells, or formation of infectious biofilms on medical implants. An example of the latter is the problematic infectious biofilms that can form on medical implants such as indwelling catheters, which are often constructed of elastomers. According to the devices, methods, and systems provided herein, problematic biofilms can be released from such catheters by subjecting their polymer surfaces to cyclic changes in surface area. The deformation of the polymer surfaces can effectively detach microbial biofilms and macro-fouling organisms.

As used herein, the term “critical strain” refers to any change in any area of the surface of the soft polymer. For example, in some embodiments where electrical actuation is applied, the surface area may change (i.e., the surface is strained/puckered), however the entire width or length of the soft polymer film does not. In other instances, the entire width and/or length may be changed, such as when the soft polymer film is stretched, pulled, twisted, etc.

In another example, the presently disclosed subject matter provides catheters and devices having a flexible, interior surface that can be deformed beyond a critical strain for debonding of a fouling agent from the interior surface when the fouling agent has bonded to the surface. The use of the term “shape” is meant in its broadest sense. For example, a change in shape as it is used herein deforms the surface beyond a critical strain for debonding of a fouling agent. A change in shape can include a change in a total surface area but such a change in total surface area is not required.

In an example, the interior surface may be a soft polymer layer that is exposed to the aqueous environment upon which the fouling agent may attach, or may be prevented from attaching. The soft polymer layer may be an inert, non-toxic and non-flammable substance. Suitable materials include, but are not limited to, polydimethyl siloxane (PDMS) or other silicone rubber, acrylic elastomer, a polyurethane, a fluoroelastomer, and the like.

The thickness of the soft polymer layer can be such that application of the actuation means will be able to cause deformation. Suitable thicknesses may be between 10 μm to 1 mm, or between 1 μm to about 500 μm. Similarly, the soft polymer layer may have a Young's modulus of between about 0.5 KPa to about 2.0 MPa, or between 1.0 KPa to about 1.0 MPa.

In certain embodiments, the soft polymer layer may be coated, such as spin coated, or coated on the rigid polymer film. In other embodiments, the outer surface of the soft polymer layer (i.e., the side facing the wetted environment) may be textured. As used herein, the term “texture” refers to any permutation of the elastomer surface that makes it not smooth, such as ridges, creases, holes, etc. In certain embodiments, the soft polymer layer comprises a corrugated surface.

In yet other embodiments, the surface of the soft polymer layer may also be modified by chemical means to further improve greater fouling resistance or fouling release. Such modifications include, but are not limited to, coating the polymer surface with hydrated polymers such as poly(ethyleneglycol)-derivatives, polyzwitterions and polymer brushes or coatings with other types of polymers, and the like.

The structure further comprises an actuation means. As used herein, the term “actuation means” refers to any means that is able to put the soft polymer layer into action or motion. In some embodiments, the actuation means may be one that applies a mechanical force to the soft polymer layer, which may be beyond the critical strain for debonding of the fouling agent. Application of this mechanical force, such as stretching, of the soft polymer layer can have an effect on the ability of fouling agents to remain adhered to the surface. Suitable mechanical forces include, but are not limited to, stretching, squeezing, twisting, shaking and the like.

The thickness of the rigid polymer layer may be between 10 nm to about 1 μm or between 1 μm to about 500 μm. Similarly, the rigid polymer layer may have a Young's modulus of between about 0.5 GPa to about 200 GPa, or between 1 GPa to about 100 GPa.

In accordance with embodiments, an active control approach is disclosed that uses inflation-generated strain of an elastomeric substrate to debond overlying biofilms. It was discovered that increasing the strain in the substrate increases the energy release rate and thereby increases the driving force for debonding of biofilm. In experiments, three-dimensional (3D) printing to fabricate proof-of-concept (POC) urinary catheter prototypes that generated enough strain to successfully debond and remove mature P. Mirabilis biofilm from their interior surfaces. The POC prototypes were less than 7 centimeters (cm) long and over 1.4 cm diameter, resulting in straining and debonding of the biofilm from only part of the surface (about 35% of the intra-luminal perimeter).

The present disclosure provides, in part, the design and optimization of a catheter (e.g., urinary catheter) for on-demand removal of biofilms from the inner luminal surface. In an aspect, the catheter utilizes multiple intra-wall inflammation lumens that are pressure-actuated to generate region-selective strains in the elastomeric urine lumen, and thereby remove overlying biofilms. In some embodiments, the intra-wall lumen includes, at least 1, 2, 3, or 4 intra-wall inflation lumens or cavities.

Catheters provided herein can generate greater than 30% strain in the majority of the luminal surface when subjected to pressure and are able to remove greater than 80% of a mixed community biofilm of p. Mirabilis and e. Coli on-demand, and furthermore able to remove the biofilm repeatedly.

Experiments using catheters disclosed herein demonstrate that biofilm debonding can be achieved upon application of both tensile and compressive strains in the inner surface of the catheter.

Further, catheters disclosed herein provide for a non-biologic, non-antibiotic method to remove biofilms and thereby for eliminating or at least reducing catheter-associated infections.

In examples, urinary catheters are provided that are capable of repeated on-demand biofilm removal. By adjusting the number and position of intra-wall inflation lumens, sufficient tensile strain is generated to debond biofilms over the majority of the internal lumen perimeter. In experiments, successive rounds of finite element modeling was utilized to optimize the predicted strain of catheter cross sectional profiles to ensure various designs fell within the fabrication capability of an industrial catheter manufacturer. Further, various prototypes with clinically relevant dimensions were made using a combination of extrusion and 3D printed reversed-mold fabrication techniques. Different materials for the prototype catheter shaft were compared to determine the ideal operational parameters for clinicians to manually inflate the built prototypes. The prototypes were characterized and their performance compared against finite element models. The prototype catheter, less than 7 mm in diameters (within the range of sizes available for clinical use) with four intra-wall inflation lumens, was able to achieve substrate strain over most of the perimeter of the main drainage lumen, as well as along the full length of the catheter. It was hypothesized that prototypes would debond a mixed community biofilm of E. coli and P. mirabilis, two of the most common bacteria found in CAUTIs, and an artificial bladder flow system was developed to grow mature biofilms inside the main drainage lumen of prototype catheters. Upon on-demand, inflation-generated actuation, the prototypes dramatically removed the vast majority of the biofilm along the full length of the catheter. After a successful biofilm removal, biofilm was regrown in the catheter, and it was demonstrated that inflation-induced strain would repeatedly remove biofilm in the catheter. Upon dissection of the catheters, it was observed that areas that underwent compressive strain, as predicted by the finite element models, debonded biofilm similarly to areas that underwent tensile strain. As discovered by these experiments, it was shown that a urinary catheter was developed that allows the repeated and thorough removal of infectious biofilms from its interior surface.

Since catheters are relatively long compared to their cross-section dimensions, the design analysis was simplified to a plane-strain problem. In analysis, the catheter designs were modeled with hybrid quadratic elements (CPE8MH) under plane-strain deformation using the software package known as ABAQUS 6.12. Pressure was applied along the inner surfaces of the inflation lumens while a free boundary condition was used along the outer surface of the catheter to predict its radial displacements. Mesh density was determined by a convergence study and 10,441 CPE8MH elements were used for the whole model. A nonlinear solution method and geometric nonlinearity were adopted in the analysis. A 0.2 mm thick wall was used between the inflation lumen (or cavity) and the main lumen for models used for selecting the number of inflation lumens. Finite elements models of the fabricated tubing used a 0.27 mm thick wall to reflect the actual dimensions achieved by the extrusion vendor. Three different materials were used for the catheters: 50 durometer silicone elastomer, 35 durometer silicone elastomer, and a more rigid sheath of 65 durometer silicone elastomer (all durometers defied per the type A scale), which were tested using a tensile tester and fitted using the Neo-Hookean model with shear modulus of 0.69 MPa, 0.52 MPa, and 2.44 MPa, respectively. The strains along the internal surface of the drainage lumens and the average radial displacement along the outer surface were calculated by the finite element model for comparison against experimental results.

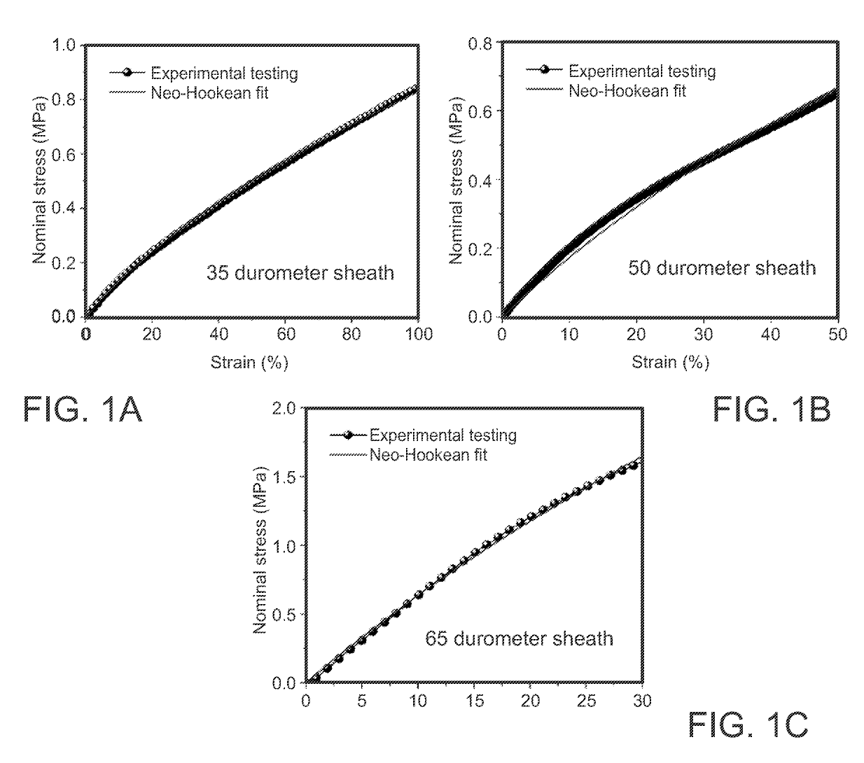

FIGS. 1A-1C illustrate graphs showing stress-strain curves for prototype materials obtained from uniaxial tensile testing. Particularly, FIG. 1A shows nominal stress-strain curves of 35 durometer silicone shaft. FIG. 1B shows nominal stress-strain curves of 50 durometer silicone shaft. FIG. 1C shows nominal stress-strain curves of “stiffer” 65 durometer silicone sheath. The curves were fit to the Neo-Hookean model. The shear moduli for the 35 durometer shaft, 50 durometer shaft, and 65 durometer sheath materials are 0.52 MPa, 0.68 MPa, and 2.44 MPa, respectively.

In experiments, extruded silicone catheter shaft components were utilized that had Dow Corning two-part, platinum-catalyzed Class VI silicone feedstock. The silicone feedstock was varied to achieve 35 and 50 durometer multi-lumen silicone main shafts and the 65 durometer silicone sheath (all durometers defined per the type A scale). In instances where a sheath was used, the sheath was slip-fitted over the main shaft using isopropyl alcohol. The inflation lumens were then sealed at each end of the main shaft using SIPPDXY® brand silicone adhesive available from Smooth-On Inc. 2 mm long holes were then skived out of the outer walls of the inflation lumen approximately 1 cm from the designed hub end of the shaft. Hub manifolds were prepared by pouring silicone (DRAGON SKIN 0020®, available from Smooth-On Inc.) into a mold prepared by a 3D printer (Dimension SST 1200ES, with patterns generated by Solidworks 20131). The inner diameter of the hubs was approximately 0.5 mm greater than the shaft in order to create a manifold to allow simultaneous inflation of all four lumens. Once cured, the hubs were removed from the molds and then pierced and fit with a male touhy borst connector to be used for inflation. The hubs were fitted over the designated hub end of the shaft and glued in the hub in place without occluding the skived holes in the inflation lumens, thus allowing simultaneous inflation of all four lumens via the touhy borst connector. Prototype performance was examined using optical video of on-end and side-views of inflation. Still images were analyzed from the video using ImageJ to characterize strain and dimensional parameters as a function of inflation pressure.

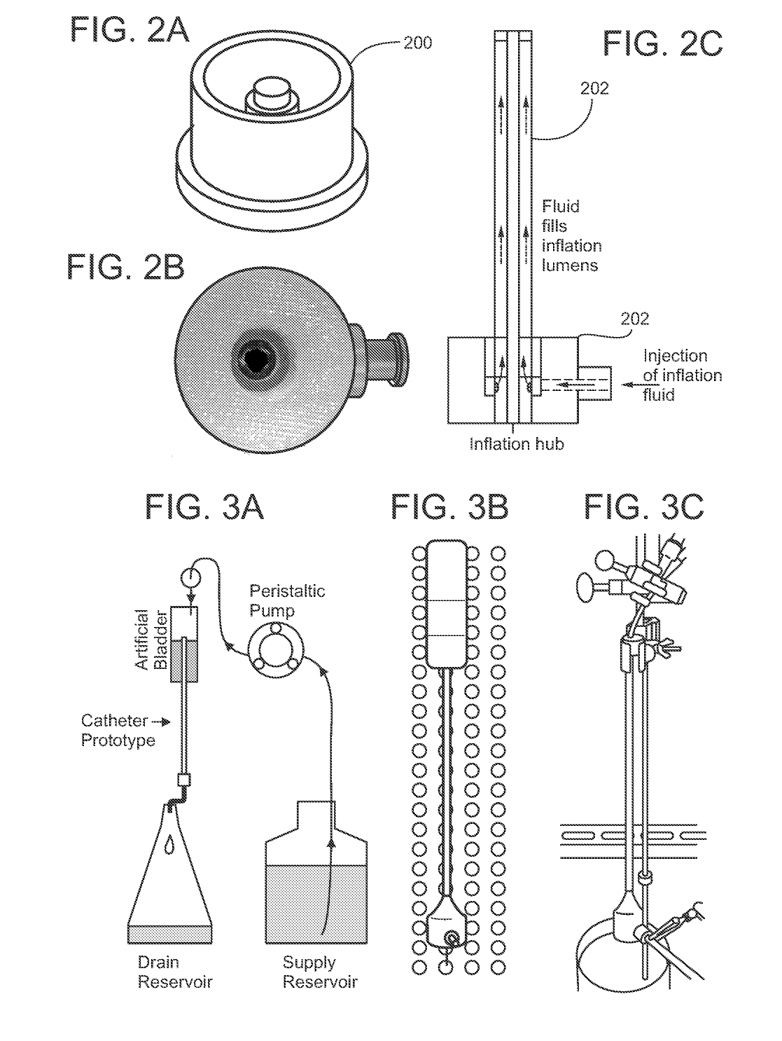

For example, FIGS. 2A-2C illustrate views of an inflation hub and its configuration with a catheter in accordance with embodiments of the present disclosure. Particularly, FIG. 2A illustrates a perspective view of an example hub mold 200 for fitting to a catheter 202 (see FIG. 2B). FIG. 2B illustrates a bottom view of the hub 202 fabricated with the mold in. FIG. 2C illustrates a cross-sectional side view of an image of an example hub fitted to an example catheter.

In experiments, Proteus mirabilis 2573 (ATCC 49565) and Escherichia coli K12 (ATCC 29425) were thawed from frozen stock and cultivated overnight at 37 degrees C. on separate tryptone soya broth agar slants which were stored at 4 degrees C. and used for up to 2 weeks. The artificial urine media formation was composed of urea 25 g/L, sodium chloride 4.6 g/L, potassium dihydrogen phosphate 2.8 g/L, disodium sulfate 2.3 g/L, potassium chloride 1.6 g/L, ammonium chloride 1.0 g/L, magnesium chloride hexahydrate 0.65 g/L, trisodium citrate dehydrate 0.65 g/L, calcium chloride 0.49 g/L, disodium oxalate 0.02 g/L, and gelatin 5.0 g/L in deionized water and was prepared. The artificial urine media was sterilized and then supplemented with 1.0 g/L tryptone soya broth prepared separately to make the total artificial urine media (AUM). Colonies of P. mirabili and E. coli were each inoculated into separate flasks of 75 mLs of AUM and grown for 4 hours at 37 degrees C. on a shaker at 240 rpm.

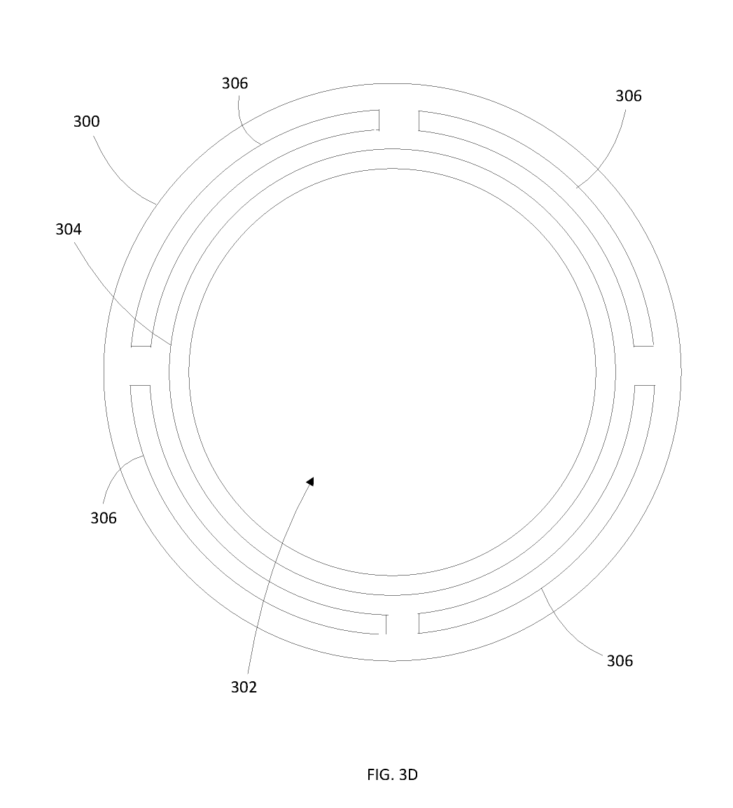

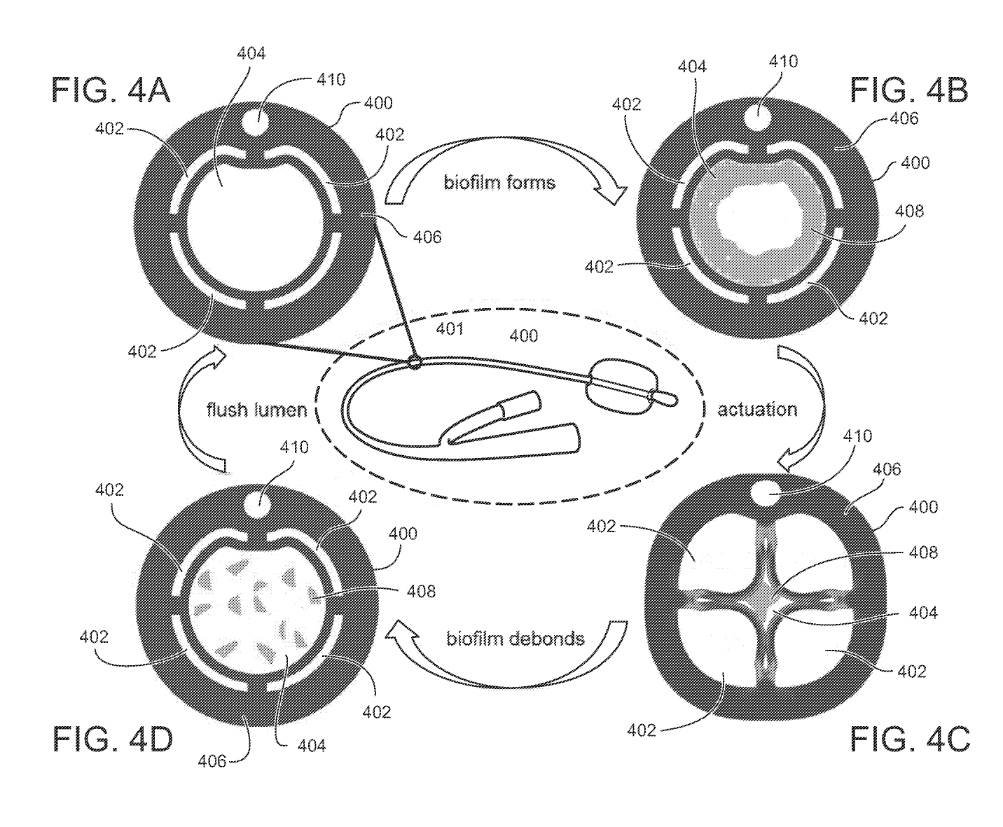

In other experiments, biofilm was grown in catheter prototypes. In particular, biofilms were grown with a co-community of P. mirabilis and E. coli on a main drainage lumen of catheter prototypes using a suitable continuous flow method. The method accommodated a manifold of four 50 mL artificial bladders in a vertical orientation. For example, FIGS. 3A-3C illustrate diagrams of example setups for biofilm-growth and debonding in urinary catheter prototypes. Particularly, FIG. 3A shows a biofilm-growth system that uses an artificial bladder to supply infected urine to the catheter. The artificial bladder is a vessel modified to accept the distal, top tip of a catheter prototype penetrating the bottom and extending approximately 4 cm into the vessel, which thereby maintains a residual volume of 30 mL in the artificial bladder. FIG. 3B shows an artificial bladder with catheter prototype with the main urine drainage lumen of the catheter prototype draining into a collection manifold on the bottom end. The diameter of the catheter prototype shaft is 6.7 mm. FIG. 3C shows a setup for rinsing and actuating to test debonding after biofilm growth.

The distal (non-hub) tips of the prototype catheters were inserted through a pressure-fit seal in the bottom of the artificial bladders. They were inserted approximately 4 cm into the bladder to ensure the bladder can hold 30 mL before draining through the catheter. The catheter prototypes, artificial bladders, and associated supply and drain tubing were sterilized and placed in a Class II biosafety cabinet. The bladders and prototypes were maintained at 37 degrees C. in a mini-incubator. The bladders each held a 30 mL reservoir of infected media that can overflow into the distal tip of the catheter prototype and then drip-feed through the main drainage lumen of the prototypes as fresh media was added to the bladder. The system was primed with AUM, and then inoculated with 4 hour cultures of 5 mL of P. mirabilis and E. coli, each introduced into the artificial bladder. The bacteria were left for 1 hour to allow attachment and infection of the bladders and catheters. The model was then run continuously at a flow rate of 0.5 mL min⁻¹ supplied via peristaltic pumping until the desired time point when a thick biofilm was visible through the walls of the prototype, or a system blockage occurred. All biofilm growth was conducted in a sterile biosafety cabinet. The sterility of the artificial bladder growth system was confirmed by control runs without bacterial inoculation; no deposition was visually observed and microscopic examination confirmed no biofilm was formed on control samples.

For examples undergoing only one round of biofilm removal, the prototypes were gently removed from the artificial bladders and kept covered in a hydrated state. The samples were suspended vertically, and artificial urine media was introduced into the upper end at a flow rate of 4 mL min⁻¹ for 1 minute. Samples designated for inflation were rapidly inflated to a pressure of 80 kPa and then deflated 10 times at 0.6 s⁻¹ to achieve 35% average strain, each inflate/deflate cycle taking less than one second, approximately 20 seconds into 1 minute rinse. At this point it was observed that the portion of the catheter shaft covered by the external manifold over-inflated, likely due to additive forces of the pressure in the manifold as well as in the inflation lumens. This over-inflation acted as a valve-like mechanism due to the over-inflation blocking more of the main lumen than blocked in the rest of the catheter shaft, and thereby reducing flow of material and fluid through the main lumen in the hub region. A catheter internal shaft was inserted into the hub portion of the catheter shaft which prevented actuation of the inflation lumens and allowed free flow of material and fluid in the main lumen through the internal shaft. Inflation was conducted hydraulically using a syringe-delivered, predetermined volume of water. Prototype samples were weighed before biofilm growth, before rinse, and after the rinse in order to assess the weight of biofilm grown and removed. The effluent from each sample's rinse was also collected. The effluent was centrifuged, the liquid supernatant, and the remaining biofilm weighed as another measure of biofilm removal. Samples were then dissected into tip, top, middle, and bottom sections. 1 mm thick sections for cross-sectional views of the main lumen and 1 cm long sections that were filleted in half for longitudinal views of the main lumen were sliced from the top, middle, and bottom sections. Those sections, in addition to cross sectional views of the tip, were then optically photographed. Image analysis to quantify the biofilm occlusion of the luminal cross-sectional area was conducted using represented images of unstained cross-sections and ImageJ version 1.49v. The image contrast was increased by 0.3% to highlight the biofilm, and the image was rendered as a binary image to show distinct areas with and without biofilm. ImageJ's area fraction measurement function was then applied to the luminal cross-sectional area. Additional pieces from the top, middle, and bottom were stained with 0.01% crystal violet for 10 minutes and rinsed 2 times with deionized (DI) water before similar slicing for cross sectional and longitudinal views. Representative longitudinal, crystal violet stained samples were carefully cut to excise the main lumen from the catheter shaft to allow flattened views of the biofilm coverage of the main lumen. Stained sections were also optically photographed, and selected sections were examined on the phase microscope at 10× magnification.

Fresh prototype catheter samples were fabricated to undergo two rounds of biofilm removal. The co-community biofilm of P. mirabilis and E. coli was grown on the main drainage lumen of catheter prototypes using the same continuous flow method described herein. Inflation actuation was utilized as disclosed herein to remove the biofilm from all samples once the biofilm formed. The consumed supply of AUM was then replaced with a fresh supply of AUM, and the drainage collection flask was emptied before re-starting the peristaltic pump at the same flow rate of 0.5 mL min⁻¹. Once the co-community biofilm regrew (after approximately 24 hours), the flow was stopped. The artificial bladders and the catheter samples were carefully removed from the flow loop and all catheters were rinsed with AUM supplied into the artificial bladder at a flow rate of 4 mL min⁻¹ for 1 minute. Samples designated for inflation were rapidly inflated to a pressure of 100 kPa and deflated 10 times to achieve 40% strain approximately 20 seconds into the 1 minute rinse. The effluent from each sample's rinse was collected and samples were then dissected as disclosed herein. Image analysis to quantify the biofilm occlusion of the luminal cross-sectional area was conducted as described herein.

Statistical comparisons were conducted using GraphPad Prism 5. Group means were compared by two-tailed, unpaired t-tests with Welch's correction to account for potentially unequal variances. “*” denotes P<0.05, “*” denotes P<0.01 and “***” denotes P<0.001 where shown in figures. Data presented as mean +/− standard deviation in bar and line graphs.

Disclosed herein are urinary catheters capable of releasing biofilms by active actuation of elastomers. For example, FIGS. 4A-4D illustrate a flow diagram of example use of a urinary catheter 401 for on-demand removal of infectious biofilms via active deformation in accordance with embodiments of the present disclosure. Particularly, FIG. 4A shows a cross-section of an end of a urinary catheter shaft 400 with intra-wall inflation lumens 402. The catheter shaft 400 is equipped with inflation lumens or cavities 402 positioned between an inner main lumen 404 and an outer catheter wall 406. FIG. 4B shows the cross-section of the end of the urinary catheter shaft 400 after biofilm 408 has formed on the interior surface of the urine drainage lumen after 1-2 days. After formation of the biofilm 408, the inflation lumens or cavities 402 can be pneumatically or hydraulically actuated to a controlled level of strain for multiple inflate/deflate cycles. FIG. 4C shows the cross-section of the end of the urinary catheter shaft 400 during actuation of inflation lumens 402 by pumping air, water, or other fluid to generate large mismatched strains between biofilm and the surface of the main lumen to debond the biofilm 408 from the urine drainage lumen 404. After multiple inflate/deflate cycles, the biofilm 408 is debonded from the interior surface of the main lumen 404 and then can be removed by a minimal flow of liquid (e.g., urine generated by a patient), thereby clearing the urine drainage lumen 404 for continued use. FIG. 4D shows the cross-section of the end of the urinary catheter shaft 400 after the detached biofilm 408 is removed by the flow of urine once the inflation lumens are deflated. As a result, the catheter 401 can be maintained free of mature biofilms for long-term use and thereby may reduce the risk of catheter-associated urinary tract infections. The lumen shaft 400 may also define a restraint balloon lumen 410 for inflating a balloon at the tip of the catheter residing in the bladder, typically as a method of securement whereby the inflated balloon is larger in diameter than the entrance of the urethra from the bladder and thereby prevents the removal of the tip of the catheter from the bladder.

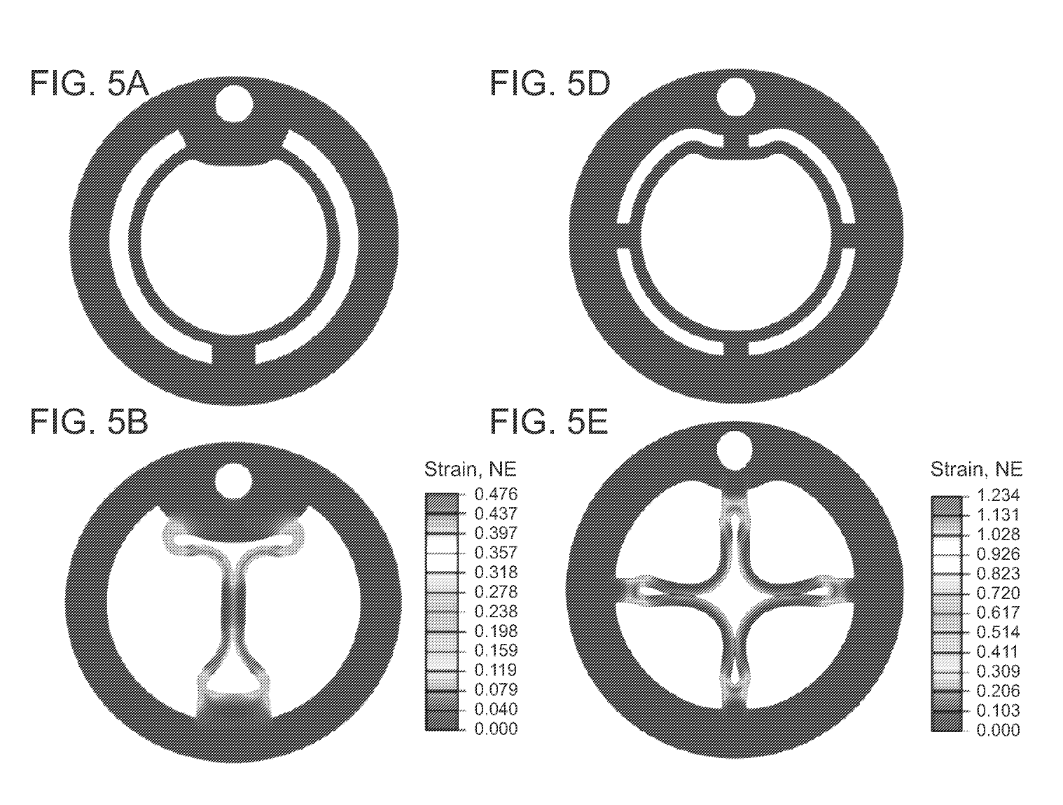

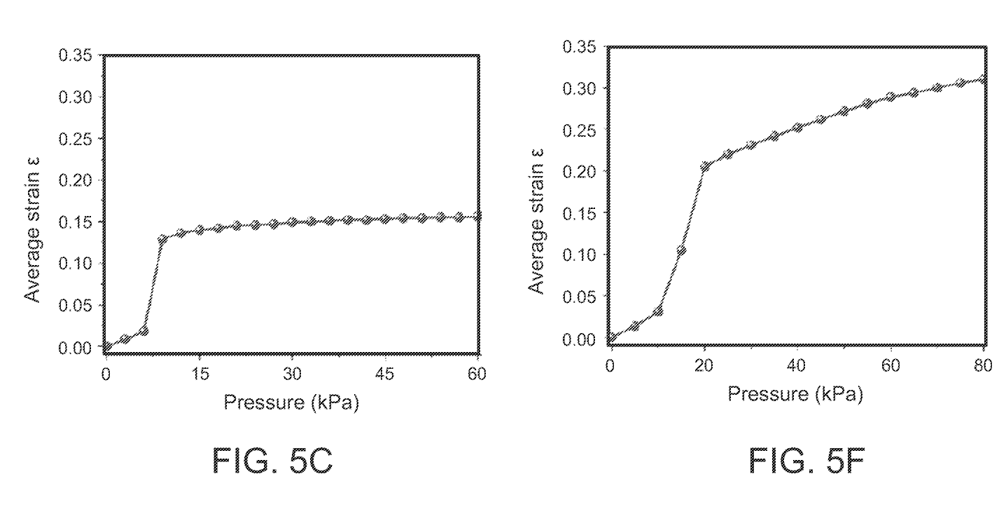

Though experiments it was discovered that active surface deformation effectively detaches mature crystalline urinary biofilms from flat and curved surfaces of silicone elastomers. Both the strain rate and strain level generated by actuation has a significant influence on biofilm debonding. The biofilm debonds once the energy release rate exceeds the adhesion strength between the biofilm and the substrate. In embodiments, catheters are designed with inflation lumens that underlie a substantial portion of the perimeter of the catheter. Finite element models were used to predict inflation performance, and the resultant strains in the wall of the main lumen. One design involved a two-inflation-lumen catheter, in which each inflation lumen occupies almost half of the perimeter of the catheter. For example, FIGS. 5A-5F illustrate finite element models and graphs in accordance with embodiments of the present disclosure. Particularly, these figures present finite element models showing that a four-inflation-lumen design for a urinary catheter shaft can achieve higher levels of tensile strains along circumferential direction in the urine luminal surface than a two-inflation-lumen design at the same inflation pressure. Referring to FIG. 5A, this figure shows a cross-section of a catheter shaft with two intra-wall inflation lumens. FIG. 5B shows predicted strains along circumferential direction in the urine luminal surface of the two-lumen catheter from finite element model when both inflation lumens are simultaneously inflated by a pressure of 60 kPA. FIG. 5C shows predicted average stain along circumferential direction in the urine luminal surface of as a function of the inflation pressure for the two-inflation-lumen configuration. FIG. 5D shows a cross-section of the catheter shaft with four inflation lumens. FIG. 5E shows predicted strains along circumferential direction in the urine luminal surface of the four-lumen catheter from the finite element model when four inflation lumens are simultaneously inflated by a pressure of 80 kPa. FIG. 5F shows predicted average strain along circumferential direction in the urine luminal surface as a function of the inflation pressure for the four-lumen configuration.

The finite element model demonstrated that, after an initial increase of the surface strain on the surface of main drainage lumn, as inflation pressure increased, the surface strain stops increasing at about 15% due to the interfering contact of the two walls in the confined space of the drainage lumen (see FIG. 5C). The biofilm debonds once the energy release rate G exceeds the adhesion strength between the biofilm and the substrate due to applied strain, and G∝μ_{f}ε²H (where μ_{f} is the storage modulus of the biofilm, ε is the applied strain in the substrate, and H is the biofilm thickness). The majority of the biofilm debonds once the applied strain in the substrate reaches a “critical” value ε_{c}. For instance, in mucoid biofilms, such as E. coli, the majority of the biofilm debonds at a critical strain of 15% (although critical strain can vary depending upon biofilm thickness and substrate modulus); and in crystalline biofilms such a P. mirabilis, the critical strain is approximately 25%. In some embodiments, the critical strain will not exceed 30%.

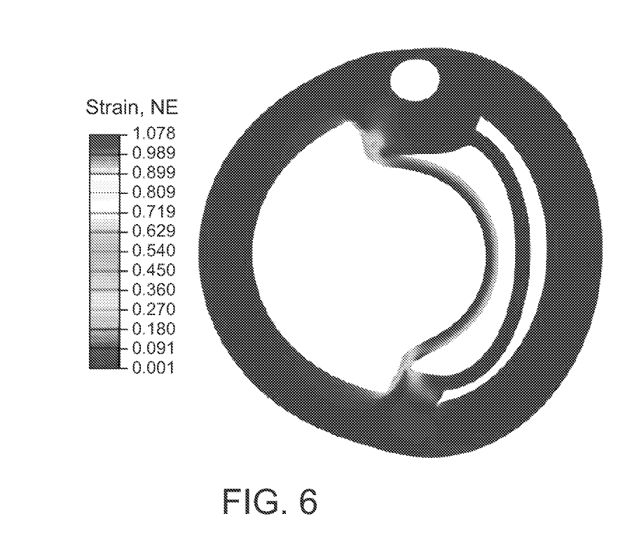

In embodiments, the lumens or cavities may be sequentially inflated to achieve desired critical strains. In some cases, this may cause significant distortion of the cross-section outer diameter as shown in the example of FIG. 6, which illustrates a contour plot of nominal strains, and the resultant deformation profile, of the cross-section of a catheter with two lumens when one lumen is actuated to achieve an average strain of 30%. Therefore, to limit interference between inflated lumens, the perimeter length of the individual inflation lumens were reduced while increasing the number of inflation lumens to four. For example, FIG. 5D shows four lumens as an example. Using finite element models as shown in the example of FIG. 5E, the strains along the internal surface of the drainage lumen reaches greater than 30% strain at a pressure load of approximate 70 kPa (assuming silicone with a shear modulus of 0.68 MPa). Healthcare practitioners can achieve 70 kPa using suitable hospital syringes.

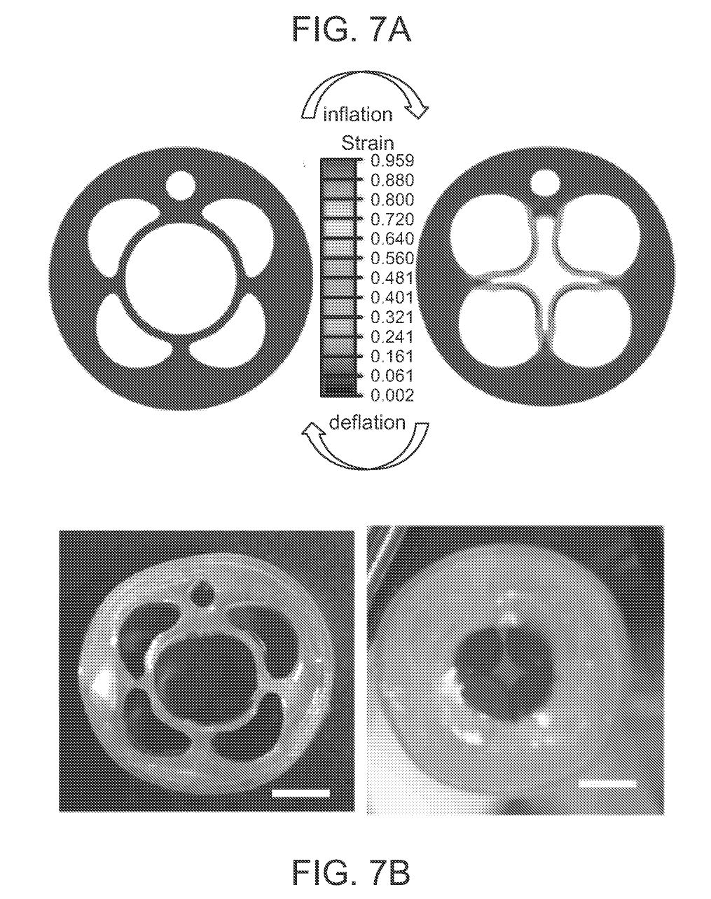

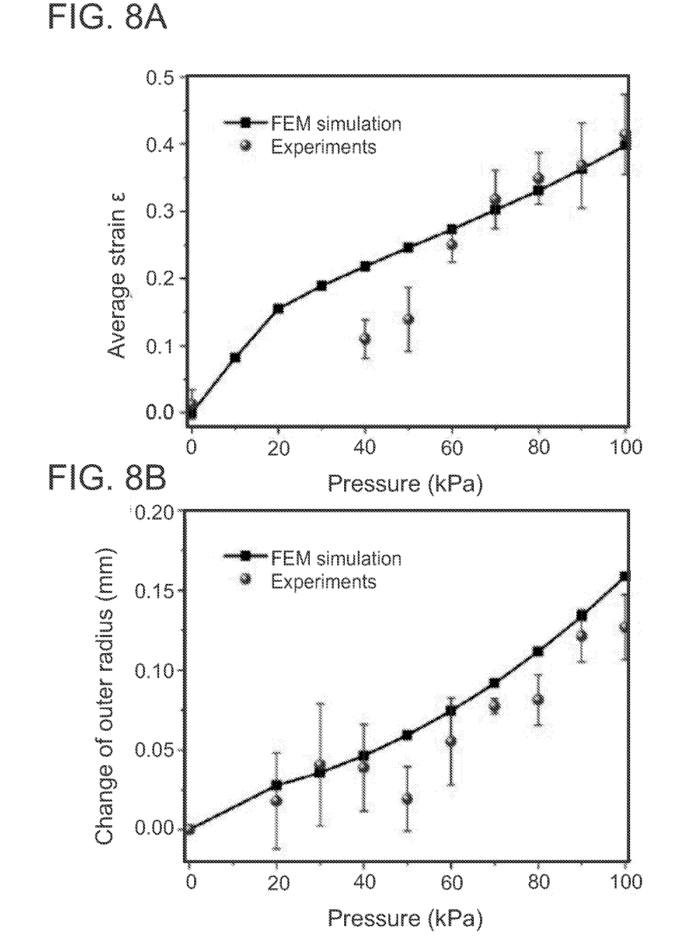

In accordance with embodiments, FIGS. 7A, 7B, 8A, and 8B show finite element analysis and experimental data of a four-lumen catheter shaft made of 50 durometer silicone elastomer. Particularly, FIG. 7A illustrates a schematic of a cross section and finite element model (100 kPa) of extruded silicone urinary catheter shaft. FIG. 7B provides photographs of the cross-section and the inflated four-lumen catheter at 80 kPa inflation pressure (the scale bar indicates 1 mm). FIG. 8A shows the average strain of the urine luminal surface of the four-lumen configuration, where the 30% strain is achieved at approximately 93 kPa. FIG. 8B shows the change of the outer radius of the shaft as a function of applied pressure. For these embodiments, catheter prototypes were fabricated using a 50 durometer silicone (Dow Corning two-part, platinum-catalyzed Class VI silicone feedstock; 50 durometer extension). In another prototype, 35 durometer catheter prototypes were fabricated. In yet another prototype, a thin-walled, higher modulus (65 durometer) “sheath” was added to the outside of the catheter to constrain the deformation of the outer surface (See FIG. 7B).

Finite element models were employed to estimate strains under different inflation pressures of the cross-section of a urinary catheter having four inflation lumens and made of low modulus silicone (35 durometer) and constrained with a high-modulus (65 durometer) silicone sheath. FIGS. 7A, 7B, 8A, and 8B show experimental testing of a catheter that agrees well with numerical prediction of strain in a central luminal surface as a function of inflation pressure. Particularly, FIG. 7A illustrates cross-section and finite element model for a silicone urinary catheter shaft with four inflation lumens. The strain contout plot in FIG. 7A represents the finite element model being subjected to an inflation pressure of 80 kPa. FIG. 3B shows a digital photograph (on the left) of the cross-section of a catheter shaft made of 35 durometer, low modulus silicone and constrained with a 65 durometer, high-modulus silicone sheath. The right image of FIG. 7B shows it profile when inflated to 80 kPa. The scale bar indicates 1 mm. FIG. 8A is a graph of calculated and experimental average strains along circumferential direction in the central luminal surface. The average strains obtained as a function of the applied hydraulic pressure are shown in FIG. 8A. The elastomer for the sheath was assumed to be a Neo-Hookean material with a shear modulus of 2.44 MPa (see FIG. 1A). FIG. 8B is a graph of the increase in the outer radius of the shaft as a function of applied inflation pressure. Simulation results confirmed that the inflated wall easily achieved substrate strains sufficient to debond crystalline biofilms (e.g., greater than 30% strain) over most of the surface (see FIG. 8A). As shown in FIG. 8B, the change in the outer radius of the shaft at higher pressure was dramatically reduced with the added sheath. The catheter sheath was experimentally actuated using colored water and verified that the numerical results agree well with experimental data in the relevant range (see FIGS. 8A and 8B) and exhibited similar appearance during the inflation process. FIG. 7B shows the deformation profile of the four inflation lumen catheter, which is similar to the profile predicted by the strain contour plot at 80 kPa as shown in FIG. 7A.

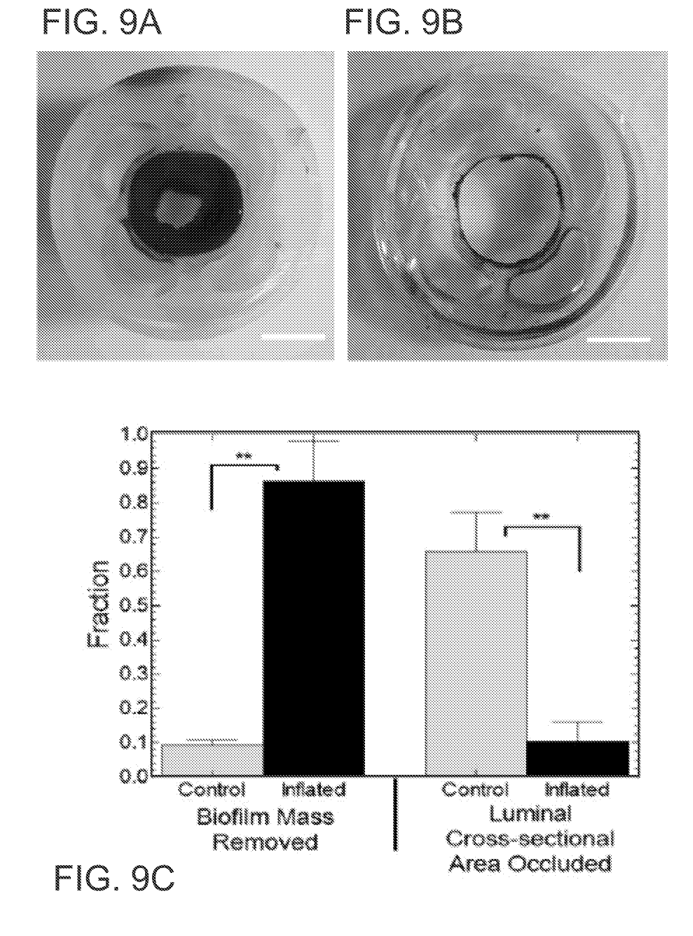

The efficacy of the new catheter in debonding a mixed community biofilm of P. mirabilis and E. coli from the main drainage lumen surface of the catheter prototype in an in vitro biofilm model. E. coli is present in up to 90% of diagnosed urinary tract infections, and P. mirabilis is another frequent infecting bacterium that can accumulate in thickness sufficiently to block the urinary catheter causing trauma, leakage, polynephritis, and septicemia, while overall being very difficult to treat. P. mirabilis and E. coli were selected to represent a difficult to remove and yet typical mixed community biofilm. The two species have been shown to be non-intering in a urinary catheter, model, so it was hypothesized that they may form a robust mixed-community biofilm. An artificial bladder biofilm growth model was modified to fit some prototypes described herein. The model fed infected artificial urine downward through prototypes (see FIGS. 3A-3C) at a rate of 0.5 mL min⁻¹, and after approximately 30 hours achieved uniform biofilm distribution around the perimeter and down the length of the main lumen (see FIG. 9A for uninflated control sample).

Once a mature biofilm was clearly visible covering the interior of the catheter, the catheters were gently removed from the artificial bladders and mounted them vertically for rinsing and testing (see FIG. 3C). Each catheter was rinsed with artificial urine media supplied at 4 mL min⁻¹ for 1 minute. Catheters designated for inflation/actuation were rapidly inflated to 80 kPa and deflated 10 times, achieving an average of approximately 35% strain, at 20 seconds into the rinse. The debonding of the biofilm due to the actuation and the subsequent removal of the biofilm in the effluent was visually observed through the walls of the catheter. In cases in which the catheter was almost clogged with biofilm, the debonded biofilm flowed downward and then re-clogged at the hub. It was realized that imperfections in the hub region were creating a choke point and thus inserted a plastic tube into the main lumen to shunt past the hub, which allowed the biofilm to flow out from the catheter in subsequent runs. The effluent was collected, centrifuged (average relative centrifugal force of 716, 5 minute duration, 22 degrees C.), removed the supernatant, and then weighed the remaining biofilm in order to quantify the biofilm detachment (biofilm mass removed normalized to the biofilm mass grown) for a particular run.

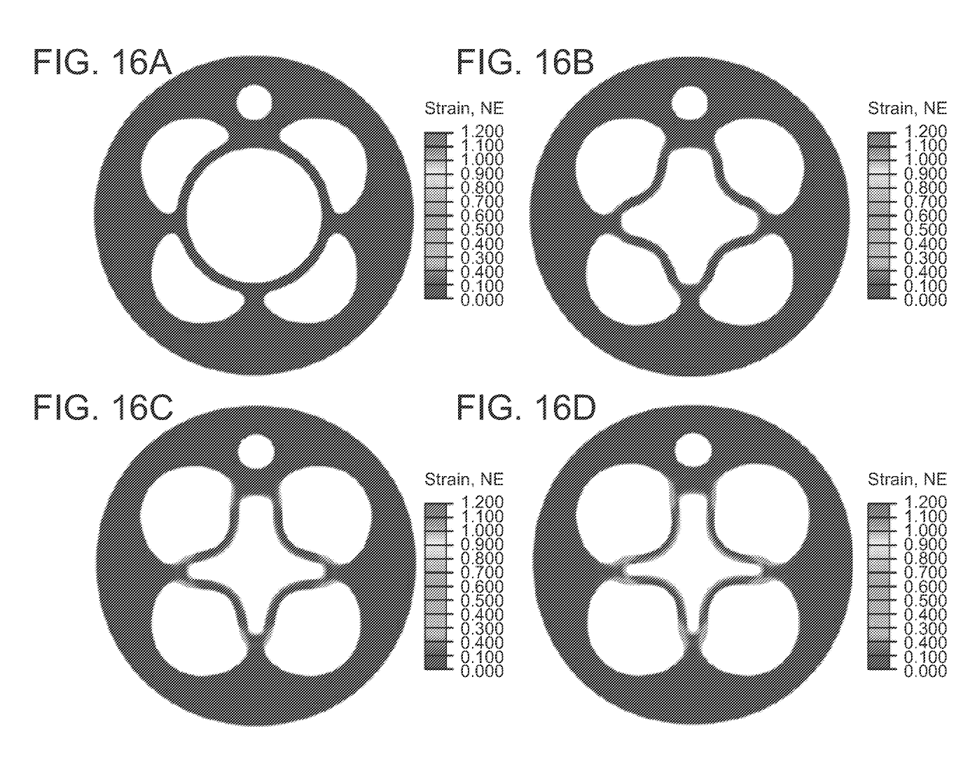

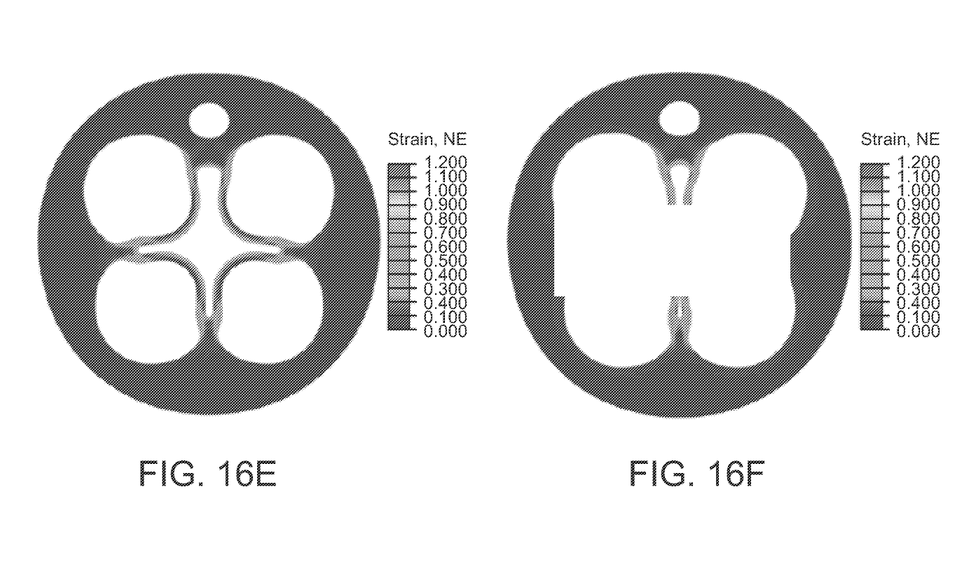

During experimentation, it has been observed that the hub region of catheters that use on-demand, inflation-generated actuation to remove biofilm can encounter over-inflation isolated to the hub region of the catheter. This can be undesirable because it temporarily creates a constriction at the “downstream” end of the catheter during on-demand inflation-generated actuation, thereby slowing down flow of urine or effluent or biofilm-laden fluid in the main lumen channel. This can additionally be undesirable because it could promote flow “upstream,” which in the example case of a urinary catheter, would suggest flow of biofilm-laden urine back into the bladder which potentially increase chance of infection rather than increase it. Therefore, it is desirable to create a solution to prevent potential over-inflation in the hub-region of a catheter, and thereby maintain the luminal space and thereby allow free flow in the hub region. As an example, FIG. 16D shows inflation around the shaft at a given pressure, while FIG. 16F shows the inflation in the hub region at the same pressure. This depicts the over inflation at the hub region, which creates constrictionat the hub region. The example catheter shown in FIG. 3D provides an example solution.

Referring to FIG. 3D, the figure illustrates a cross-sectional end view of a catheter 300 as configured within a hub (not shown for ease of illustration) in accordance with embodiments of the present disclosure. The catheter 300 includes an inner main lumen 302 surrounded by a rigid tubular structure 304. The tubular structure 304 extends at least along a length of the lumen 302 that is within the hub. Surrounding the rigid tubular structure 304 are multiple inflation lumens or inflation cavities 306. When pneumatically or hydraulically actuated as disclosed herein, the inflation lumens 306 can apply pressure towards the main lumen 302. The rigid tubular structure 304 provides resistance to the applied pressure for preventing the inflation lumens 306 from overinflating. During experimentation, it was observed that inclusion of the tubular structure 304 allowed for suitable inflation of the rest of the catheter while preventing constriction of the main lumen 302 within the hub.

Finally, the catheter was removed and sectioned to facilitate observation of the biofilm on the main drainage lumen surface. Sections from the top, middle, and bottom of the catheter shaft were also stained with 0.1% crystal violet to enhance biofilm visualization. FIGS. 9A-9C show representative optical images of the cross sections of control urinary cathether shaft with mixed community P. mirabilis and E. coli biofilm intact on the main lumen of a control versus an actuated catheter. As shown in the representative images, the majority of the biofilm accumulated in the main lumen was clearly removed by inflation. The normalized biofilm mass removed was statistically analyzed, and it was confirmed that the inflation removed a large fraction (about 80%) of P. mirabilis and E. coli biofilm mass (p<0.005 for N=3 replicates). Representative unstained cross sections from each catheter were also analyzed for the fraction of luminal cross sectional area occluded by biofilm, and the image analysis confirmed that little biofilm remained in the lumen of inflated samples (p<0.01, see FIG. 9C).

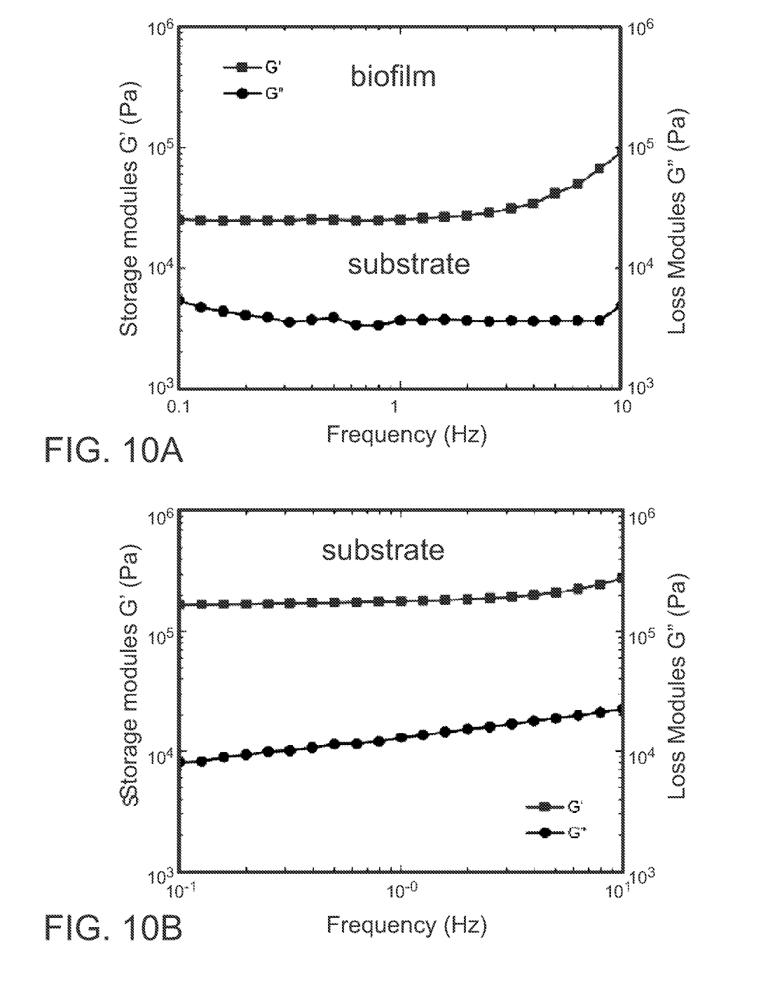

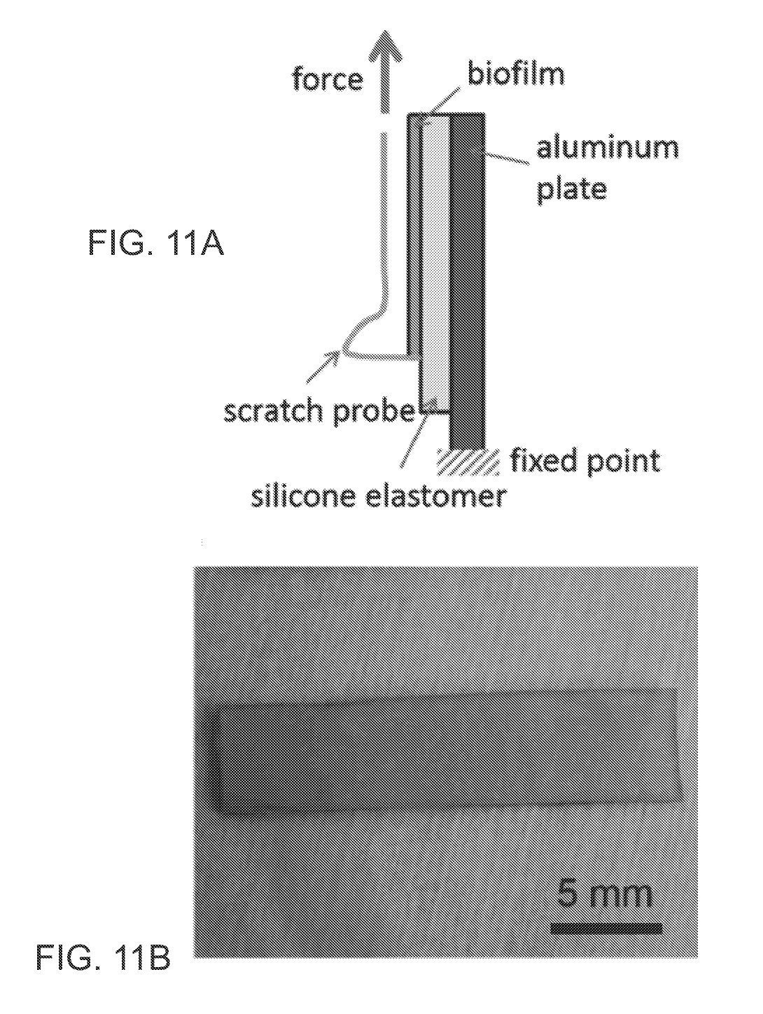

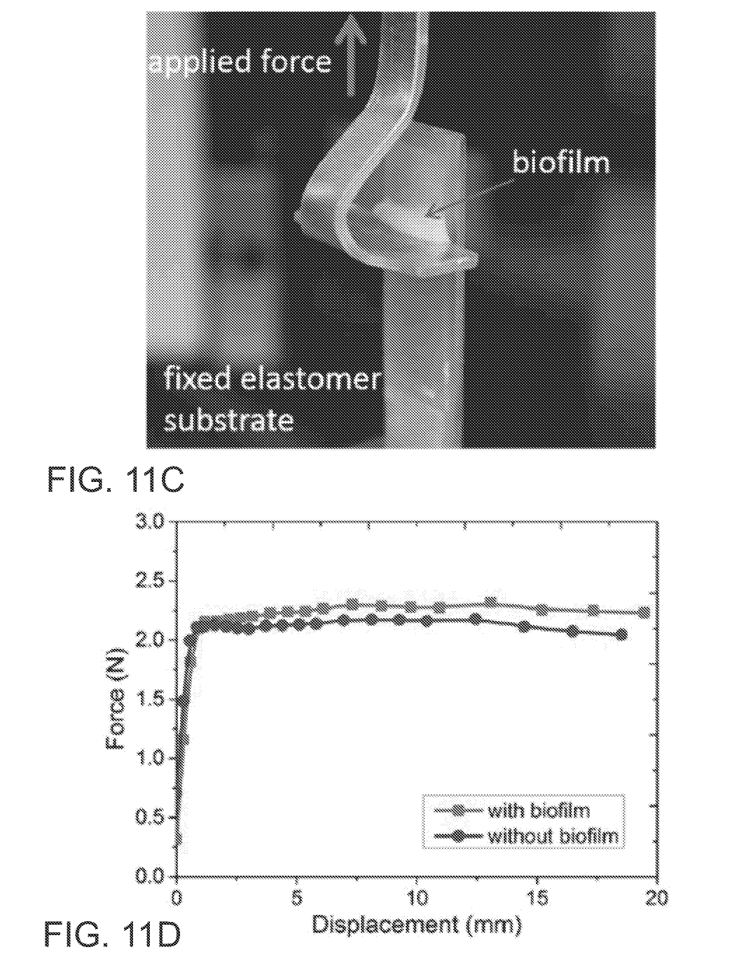

It was observed that the biofilm exhibited a predominantly crystalline composition. In order to analyze the mechanical properties of the co-biofilm, the mixed community biofilm was grown on flat silicone samples. The complex visco-elastic modulus of the biofilms was tested using an AR G-2 Rheometer. The mixed community biofilms of P. mirabilis and E. coli were predominantly elastic with a storage modulus G′ of about 2.5×10⁴ Pa and loss modulus G″ of about 3.9×10³ Pa for the scanned frequencies (see FIG. 10A). The adhesion strength of the biofilm was tested based on a modified scratch test (see FIGS. 11A-11D) and found that the co-biofilm exhibited an adhesion strength of approximately about 8 J m⁻².

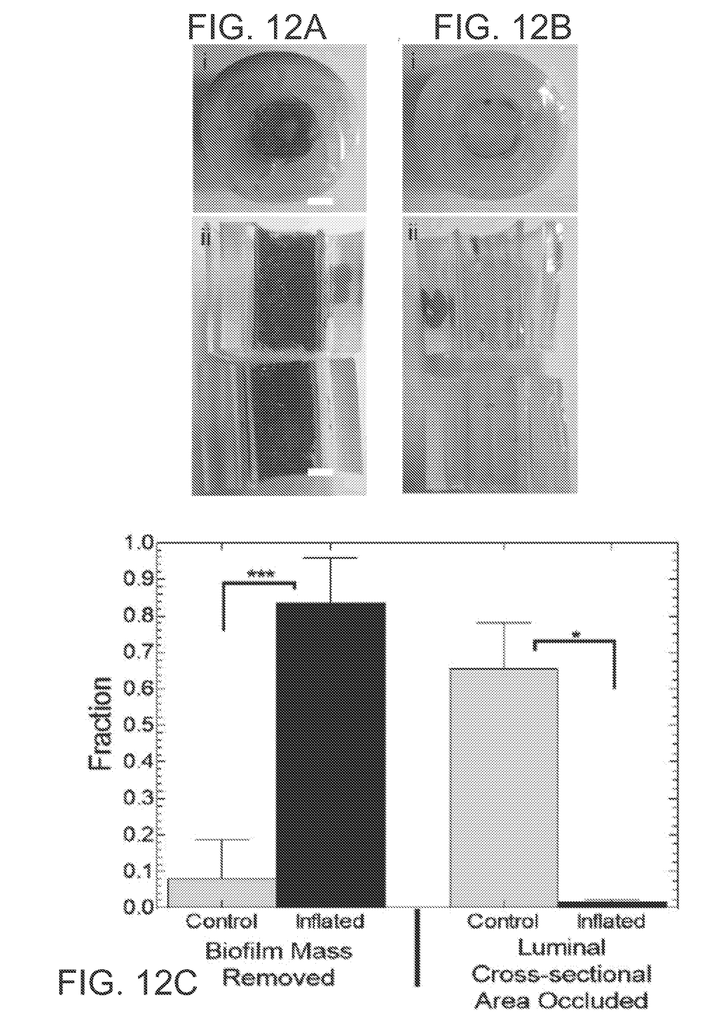

To assess the performance of the catheter in repetitive debonding of the biofilm for long term use, biofilm was regrown using the artificial bladder system for 24 hours after initially debonding the biofilm from all of the sample catheters after about 30 hours of biofilm growth. The catheter prototypes “in situ” were left in the artificial bladders during the rinse and debonding steps to more closely simulate clinical conditions. Artificial urine media accumulated in the artificial bladders before flowing into the distal tip of the cather instead of being fed directly into the distal tip. Catheters designed for inflation after the second round of biofilm growth were rapidly inflated to 100 kPa (approximately 40% strain) and deflated 10 times approximately 20 seconds into the rinse. Biofilm debonding was observed from the main drainage lumen upon inflation actuation. The biofilm effluent was collected and weighed in the rinse effluent. FIG. 12A describes the performance of the second run of debonding after re-growing the biofilm; actuation again removed the majority of the mixed community biofilm (83.6+/−6.2%, N=4) at a statistically significant level (p<0.001). The prototypes were removed, sectioned, and optically imaged. FIGS. 12A and 12B show the representative optical images from cross sections that were crystal violet stained to enhance visualizations. Biofilms were re-grown on samples that had undergone actuation. Samples were rinsed at 4 mL min⁻¹ of artificial urine for 1 minute. Catheters designated for inflation were inflated to 100 kPa (approximately 40% average strain) 10 times. Sections of the catheter shaft were removed and imaged, and select sections were crystal violet stained to enhance biofilm visualization. FIG. 12A shows representative optical images from control samples (no inflation); both (i) cross section, and (ii) sliced open samples show thorough biofilm coverage. The scale bar indicates 1 mm. FIG. 12B shows representative optical images from inflated samples; (i) both cross section, and (ii) sliced open samples show substantial biofilm removal. FIG. 12C is a graph showing that inflation removed a significant fraction of re-grown biofilm mass in each run (N=4 replicates). Likewise, biofilm occluded a large fraction of control samples' luminal cross-sectional area, but was removed from inflated samples (N=3 replicates). “***” indicates p<0.001 and “*” denotes p<0.05. Control samples show thick biofilm coverage and inflated samples confirm substantial biofilm removal. Unstained cross sections from each catheter were used to assess the fraction of luminal cross sectional area occluded by biofilm, and confirmed that little biofilm remained in the lumen of inflated samples (about 1.9%, p<0.05, see FIG. 12C).

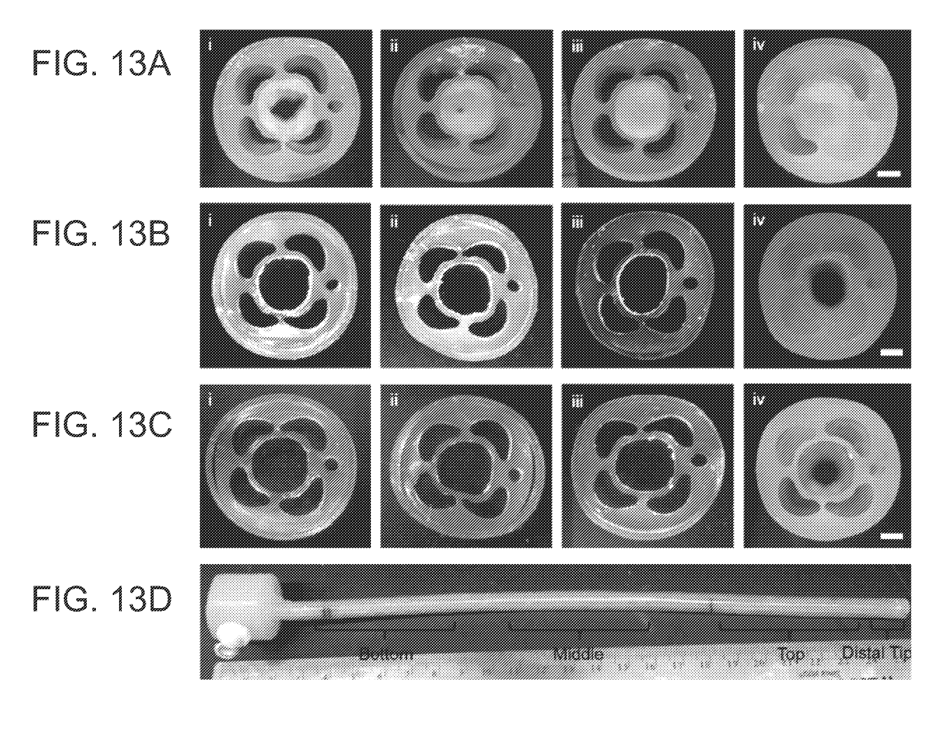

FIGS. 13A-13D show optical images of cross sections along the length of three representative urinary cathers' shafts; a control, a catheter that underwent one round of biofilm debonding, and a catheter that underwent two rounds of biofilm debonding. Biofilm removal clearly occurs along the length of the catheter, thereby confirming that the intra-wall actuation works along the length of the catheter. Additionally, the second round of biofilm removal appeared to be just as successful at removing biofilm as the first actuation. Referring to the figures, FIG. 13A shows a control catheter with no inflation, FIG. 13B shows a first round of inflation after 30 hours of growth of biofilm, and FIG. 13C shows a second round of debonding after re-growing the biofilm for another 24 hours. FIG. 13D shows sections taken from the prototypes at the following locations: bottom, middle, top, and distal tip. The scale bars indicate 1 mm.

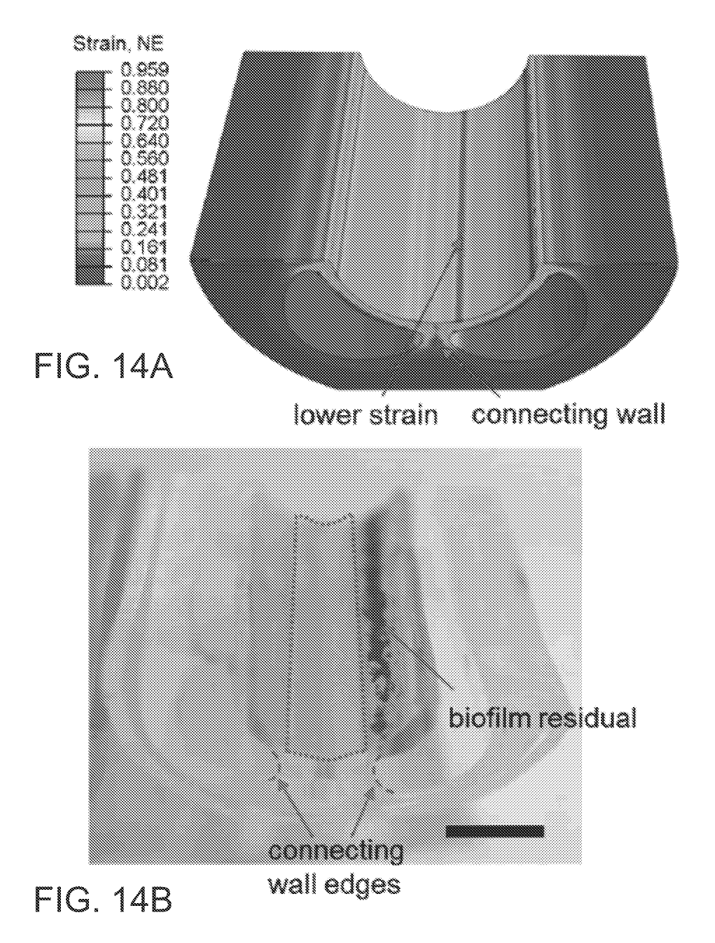

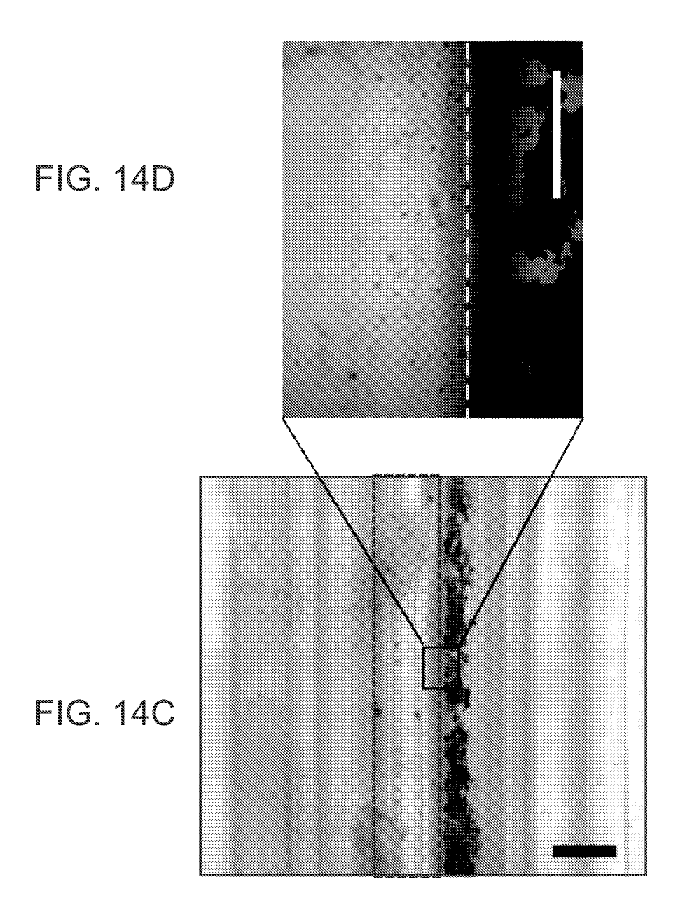

Tensile strain to the substrate can debond overlying biofilm from the substrate. FIG. 14A shows the strain predicted by finite element models to have occurred in a catheter inflated to 100 kPa, and maps the absolute value of the strain onto the surface of the catheter after deflation. The area of the luminal surface overlying the wall between intra-wall inflation lumens (i.e., the connecting wall) does not undergo tensile strain, but does undergo a significant amount of compressive strain. The area of the luminal surface that undergoes the least strain is the very edge of the intra-wall inflation lumen, where the strain transitions from tensile to compressive and presents as an area of low absolute strain. In inspection, the areas of the luminal surface that had undergone compressive strain still had debonded the majority of the biofilm. The luminal surface was excised from the rest of the catheter shaft in representative samples and captured optical images (see FIG. 14C) and microscope images (see FIG. 14D) and confirmed that the biofilm was removed in areas of high compressive strain, and residual biofilm was at the predicted edge of the inflation area where low strain values were predicted.

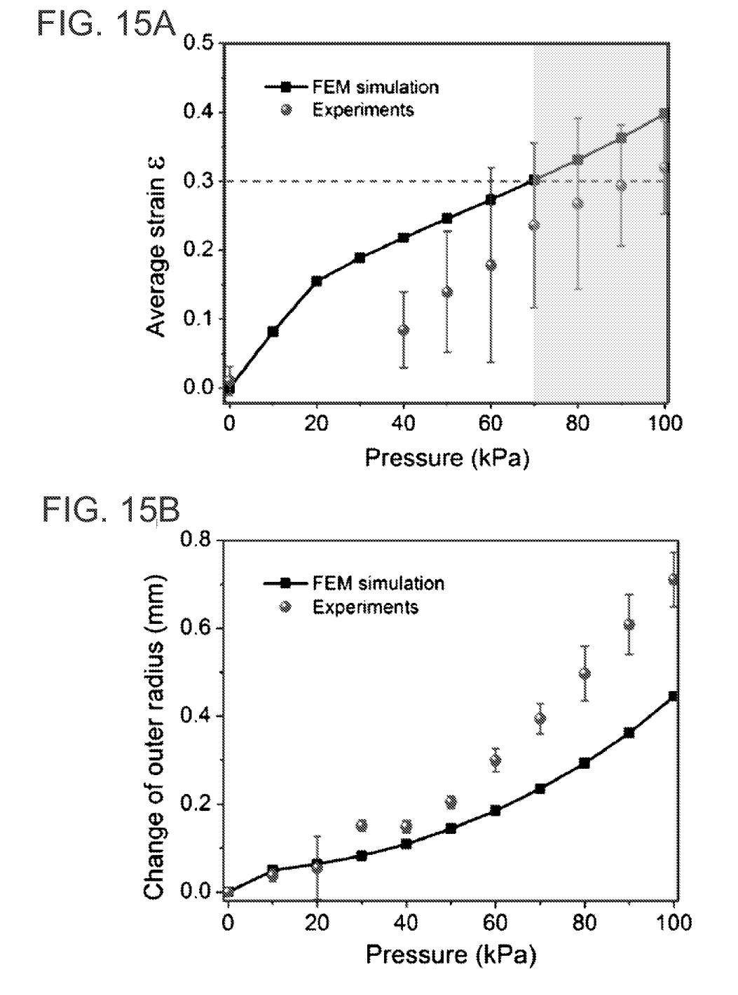

FIGS. 15A and 15B are graphs showing finite element analysis and experimental data of an extruded four-lumen catheter shaft made of 35 durometer silicone elastomer. FIG. 15A shows the average strain of the luminal surface for the four-lumen configuration, where 30% strain is achieved at approximately 70 kPa. FIG. 15B shows the change of the outer radius of the shaft as a function of applied pressure.

FIGS. 16A-16F show deformation profiles from a finite element model of an extruded four-lumen catheter shaft made of 35 durometer silicone shaft and with a 65 durometer silicone sheath when it is subjected to a range of pressures. FIGS. 16A-16F show, respectively, deformation profiles predicted at the following pressures: 0 kPa, 20 kPa, 40 kPa, 60 kPa, 80 kPa, and 100 kPa.

Various versions of the catheter were generated. Initially, finite element models were created that had 50 durometer silicone with a shear modulus of 0.68 MPa. From the finite element analysis (see FIG. 7C), it was predicted that a hydraulic pressure greater than 95 MPa would be needed to obtain the critical strain of 30% in the manufacturable four-inflation-lumen design. FIG. 7A shows the contour plot of the strains in a catheter shaft composed of 50 durometer silicone and subjected to a pressure of 100 kPa. A majority of the perimeter of the main lumen reached a high strain level of above 30% when subjected to 100 kPa pressure. FIG. 7B shows the cross-section of the obtained catheter tubes fabricated using the 50 durometer elastomer. Once inflation hubs were attached to the prototype catheter shafts, the shafts' strain upon hydraulic actuation was characterized. The experimental inflation pressure had to be higher than the predicted pressure to achieve a satisfactory strain level (see FIGS. 7A and 7B).

Catheter prototypes constructed using the 50 durometer silicone shaft required inflation greater than 100 kPa to achieve the desired substrate strain. Another extrusion with a “softer” 35 durometer silicone feedstock (shear modulus of 0.52 MPa) was conducted rather than the 50 durometer feedstock. FIG. 15A presents the average strain of the 35 durometer catheters obtained under various inflation pressures. It was found that the pressure to achieve a 30% average strain because only 70 kPa (see FIG. 15A). However, the increase in outer radius from both simulations and experiments becomes much larger (as compared to the catheter with 50 durometer elastomer, see FIGS. 7D and 15B), and rose to an unacceptable level.

In order to assess the mechanical properties of the mixed community biofilm, biofilms were grown on flat surfaces that were conducive to mechanical property characterization. Two-part silicone were poured and allowed to set to generate flat silicone samples that were trimmed to 24 mm×75 mm to fit in a drip flow reactor. The flat samples were sterilized in the biosafety cabinet by rinsing with 95% ethanol and sterilized water. The drain of a drip flow reactor was modified to keep the flat silicone coupons submerged in 0.3-0.6 cm media while under flow. The reactor was maintained at 37 degrees C by placing it in a mini-incubator. AUM was introduced using a peristaltic pump to prime the flow system. The samples in the reactor were infected with 4 hour cultures of 5 mL each of P. mirabilis and E. coli, and the infected culture was left for 1 hour to allow bacterial attachment before the media supply was resumed. The reactor was run continuously at a flow rate of 0.5 mL min' until the desired time point, or until a system blockage occurred.

Once the mixed community biofilm had formed on the flat silicone coupons, they were removed from the reactor. Smaller silicone samples (10×10 mm) were carefully cut from the silicone coupons and then used to perform a frequency-sweep oscillation test at room temperature in a mechanical rheometer. It was assumed that the biofilm did not flip during the testing process. Bare silicone samples were measured as controls. The applied strain amplitude for testing was 0.5%, and the frequency was swept from 0.1 to 10 Hz. The measured storage and loss moduli for the biofilm and the substrate are presented in FIGS. 10A and 10B.

Biofilm adhesion was tested. The adhesive strength between the biofilms and the silicone substrate is defined as the work per unit area required to remove the biofilms from the surface. As shown in FIGS. 11A-11D, a rake-shaped aluminum probe with a width of 1 cm was fabricated for the scratch testing. The biofilm-covered silicone sample was affixed at the bottom with the grip. The probe was adjusted to penetrate into the biofilm and slightly touch the silicone substrate. The probe was then moved at a controlled rate (0.5 mm/s) to scrape the surface of the biofilm-covered sample. Thereafter, a following run on a biofilm-free substrate was performed as a control. FIGS. 11A-11D show the shear forces measured for a control and an experiment. The adhesion strength between the biofilm and the silicone substrate can be calculated using measured forces and sample dimensions.

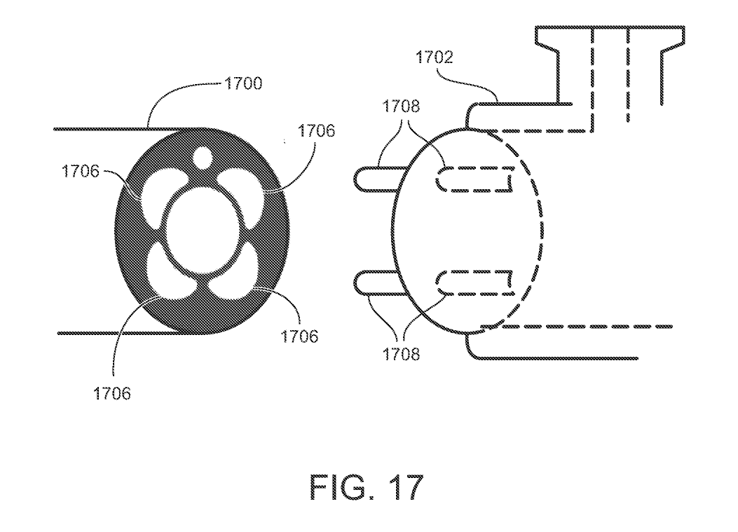

FIG. 17 illustrates an end view of an example lumen shaft 1700 and a mating manifold 1702 in accordance with embodiments of the present disclosure. Referring to FIG. 17, the shaft 1700 includes a main lumen 1704 and multiple inflation lumens 1706. The manifold 1702 includes male features 1708 that can insert into the inflation lumens 1706. Each male feature 1708 has an inflation throughway that communicates pressure between the inflation lumens 1706 and the manifold 1702. A hub or manifold such as this that mates end-on with the inflation lumens 1706 of the tubing does produce over-inflation of the inflation lumens in the manifold region since it does not have a pressurized manifold surrounding the shaft of the tubing as in the manifold depicted in FIG. 2. The manufacturing of such a manifold can be more difficult due to the small size of the male features 1708, the positioning of the male features 1708 into the inflation lumens 1706, and bonding of male features 1708 and manifold 1702 in such a way that prevents communication of pressure or fluid to the main lumen 1704.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims. One skilled in the art will readily appreciate that the present subject matter is well adapted to carry out the objects and obtain the ends and advantages mentioned, as well as those inherent therein. The present examples along with the methods described herein are presently representative of various embodiments, are exemplary, and are not intended as limitations on the scope of the present subject matter. Changes therein and other uses will occur to those skilled in the art which are encompassed within the spirit of the present subject matter as defined by the scope of the claims.

What is claimed:

1. A catheter comprising:
a lumen defining a flexible, interior surface that extends substantially along a length of the lumen for contacting a biological material;
a plurality of cavities extending along the length and positioned within the lumen adjacent to the surface, wherein the cavities each define a cavity opening; and
an inflation hub defining hub openings connected to respective cavity openings, the inflation hub defining a pump port configured to interface with a pump, the inflation hub defining at least one fluid pathway that extends between the hub openings and the pump port for permitting flow of gas between the pump and the cavities.

2. The catheter of claim 1, wherein the lumen is made of a polymer.

3. The catheter of claim 1, wherein the lumen is made of one of polydimethyl siloxane, silicone rubber, acrylic elastomer, polyurethane, and fluoroelastomer.

4. The catheter of claim 1, wherein the cavities are configured to be inflated to respective shapes for causing at least a portion of the interior surface to deform beyond a critical strain for debonding of a fouling agent from the interior surface when the fouling agent has bonded to the surface.

5. The catheter of claim 1, wherein inflation of the cavities to the respective shapes causes application of mechanical forces to the interior surface for changing the interior surface from a first shape to a second shape.

6. The catheter of claim 1, wherein the cavities substantially surround the interior surface and are configured to be inflated and deflated such that the cavities impinge on the interior surface when inflated to change the interior surface from the first shape to the second shape, and when the cavities are deflated to change the interior surface back to the first shape.

7. The catheter of claim 1, wherein the cavities are fluidly connected to the pump port via the inflation hub.

8. The catheter of claim 1, wherein the pump is configured to inflate the cavities via application of pneumatic pressure.

9. The catheter of claim 1, wherein the lumen includes a first end and a second end, and wherein the cavity openings are positioned at the first end.

10. The catheter of claim 1, further comprising a rigid structure positioned between the lumen and cavities.

11. The catheter of claim 10, wherein the rigid structure is tubular in shape.

12. A method for debonding a biological material from a catheter, the method comprising:
providing a catheter comprising:
a lumen defining a flexible, interior surface that extends substantially along a length of the lumen for contacting a biological material;
a plurality of cavities extending along the length and positioned within the lumen adjacent to the surface, wherein the cavities each define a cavity opening; and
an inflation hub defining hub openings connected to respective cavity openings, the inflation hub defining a pump port, the inflation hub defining at least one fluid pathway that extends between the hub openings and the pump port for permitting flow of gas between the pump and the cavities; and
applying the flow of gas into the pump port for inflating the cavities.

13. The method of claim 12, wherein the lumen is made of a polymer.

14. The method of claim 12, wherein the lumen is made of one of polydimethyl siloxane, silicone rubber, acrylic elastomer, polyurethane, and fluoroelastomer.

15. The method of claim 12, wherein the cavities are configured to be inflated to respective shapes for causing at least a portion of the interior surface to deform beyond a critical strain for debonding of a fouling agent from the interior surface when the fouling agent has bonded to the surface.

16. The method of claim 12, wherein inflation of the cavities to the respective shapes causes application of mechanical forces to the interior surface for changing the interior surface from a first shape to a second shape.

17. The method of claim 16, wherein the cavities substantially surround the interior surface and are configured to be inflated and deflated such that the cavities impinge on the interior surface when inflated to change the interior surface from the first shape to the second shape, and when the cavities are deflated to change the interior surface back to the first shape.

18. The method of claim 17, wherein the cavities are fluidly connected to the pump port via the inflation hub.

19. The method of claim 12, wherein the lumen includes a first end and a second end, and wherein the cavity openings are positioned at the first end.

21. The method of claim 12, further comprising providing a rigid structure positioned between the lumen and cavities.

22. The catheter of claim 21, wherein the rigid structure is tubular in shape.
